(19) United States
(12) Patent Application Publication
(10) Pub. No.: US 2024/0301399 A1
(43) Pub. Date: Sep. 12, 2024

(54) MICROFLUIDIC CONCENTRATION AND BUFFER EXCHANGE APPARATUSES AND METHODS

(71) Applicant: Nutcracker Therapeutics, Inc., Emeryville, CA (US)

(72) Inventors: Benjamin Eldridge, Danville, CA (US); Ximiao Wen, Hayward, CA (US); Ming C. Wu, Moraga, CA (US)

(21) Appl. No.: 18/276,169

(22) PCT Filed: Feb. 8, 2022
(86) PCT No.: PCT/US2022/015584

§ 371 Date: Aug. 7, 2023

Related U.S. Application Data
(60) Provisional application No. 63/147,229, filed on Feb. 8, 2021.

(51) Int. Cl. C12N 15/10; B01D 63/08

(52) U.S. Cl. CPC C12N 15/1017; B01D 63/088; B01D 2313/04; B01D 2315/10

(57) ABSTRACT

Microfluidic apparatuses including concentrators and buffer exchange regions that concentrate and exchange buffer. Also described are methods of passing a solution through a feed channel, filtering small molecules out of the feed channel by tangential flow filtration into a permeate channel adjacent to the first feed channel while maintaining a constant sheer rate relative to the membrane separating the feed channel from the permeate channel and exchanging buffer into the solution and concentrating the solution in a second region of the apparatus.

### PRIORITY CLAIM

This patent application claims priority to U.S. provisional patent application No. 63/147,229, titled “MICROFLUIDIC CONCENTRATION AND BUFFER EXCHANGE APPARATUSES AND METHODS” and filed on Feb. 8, 2021, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Currently available technologies for manufacturing and formulating polynucleotide therapeutics, particularly mRNA therapeutics, often expose the products to contamination and degradation. Currently available centralized production can be too costly, too slow, and susceptible to contamination for use in therapeutic formulations possibly including multiple polynucleotide species. Development of scalable polynucleotide manufacturing, production of single patient dosages, reduction (or in some cases elimination) of touchpoints to limit contamination, input and process tracking for meeting clinical manufacturing requirements, and use in Point-of-Care operations can advance the use of these promising therapeutic modalities. Microfluidic instrumentation and processes can provide major advantages against these goals.

### SUMMARY OF THE DISCLOSURE

The apparatuses and methods described herein may be used for the manufacture and formulation of biomolecule-containing products, particularly therapeutic polynucleotides including, but not limited to, mRNA-based therapeutics (e.g., mRNA-based vaccines). In particular, described herein are methods and apparatuses for concentrating and exchanging the buffer of a therapeutic polynucleotide.

In general, described herein are apparatuses and method for formulating composition using microfluidic apparatuses. In particular, described herein are methods and apparatuses that include processing a therapeutic biomaterial (e.g., a therapeutic polynucleotide, such as a therapeutic mRNA) using a microfluidic apparatus. These apparatuses and methods may concentrate a therapeutic biomolecule solution to an acceptable volume for the administration of the therapeutic biomolecule has been formulated. These same apparatuses may also exchange the buffer, to change the buffer for the therapeutic biomolecule solution post-mixing and post concentration to a more biocompatible and stable buffer for storage and injection into patients. For example, these methods and compositions may, as part of a single microfluidic apparatus or apparatus component, concentrate and exchange buffer to reduce the ethanol concentration and/or the concentration of other components used during formulation that are not desirable in the final therapeutic composition. The buffer within the composition may be changed to improving the long-term stability of therapeutic particles. As used herein a therapeutic may be referred to as a therapeutic composition, therapeutic particles, a drug, or drug particles, and may include a therapeutic RNA that includes an RNA molecule at least partially encapsuled by a delivery vehicle composition. The RNA herein may be an mRNA. The therapeutic may take the form of, for example, nanoparticle.

The methods and apparatuses described herein may apply a modified form of a single pass tangential flow filtration process in which an ultrafiltration membrane is used to separate the biomolecules (e.g., therapeutic polynucleotides, which may be encapsulated in a nanoparticle) from a solvent used during the formulation. Extra solvents may pass through the membrane to waste, while concentrated therapeutic particles in the same solvent may be collected downstream. Buffer exchange may be done by adding (e.g., diluting with) new buffer and concentrating either sequentially or concurrently. In some examples, buffer exchange may be done by removing old buffer from the therapeutic solution and adding new buffer to the therapeutic solution at the same time.

For example, described herein are microfluidic apparatuses, the apparatuses comprising: a first concentrator region comprising: a first permeate channel that extends in a first serpentine pathway in a first layer, a first feed channel that extends in the first serpentine pathway in a second layer from a feed input to a first retentate output, wherein the first permeate channel extends adjacent to the first feed channel, and a first membrane region that separates the first permeate channel from the first feed channel; a buffer input downstream the first retentate output for adding buffer into a retentate leaving the first feed channel; and a second concentrator region in fluid communication with the first retentate output. These apparatuses may also include one or more flow rate sensors (flow sensors), pressure sensors, and valves.

The cross-sectional area of first feed channel may decrease along the first serpentine pathway from the feed input to the first retentate output to maintain a constant sheer rate within the first feed channel. For example, a height of the first feed channel may shorten along the first serpentine pathway from the feed input to the first retentate output.

Any of these apparatuses may include a sealing structure comprising, for example, a pressure-distributing seal that distributes pressure to seal the first membrane region between the first layer and the second layer. For example, the sealing structure may be a pressure-distributing seal that may comprise a sheet of compressive foam. In some examples, the pressure-distributing seal comprises a pressurizing chamber.

The first serpentine pathway may have a length of greater than about 5 meters. The first membrane region may have an area of greater than about 50 cm². In some examples, the first membrane region comprises membrane comprising an organic material. For example, the membrane may be a Polyethersulfone (PES), a Composite Regenerated Cellulose (CRC) membrane, or a combination thereof. Thus, the first membrane region may have a high permeability for ethanol. In some examples. the membrane is chosen for high permeability for ethanol, buffer, and small molecules (e.g., DNA, RNA, lipids and etc.). In addition, the membrane may be chosen for low binding to the drug particles to minimize material loss.

Any of the apparatuses (e.g., devices, systems, etc.) described herein may include a feed input port fluidly connected with the feed input and a retentate output port fluidly connected with the second concentrator.

Also described herein are microfluidic concentrator and buffer exchange apparatuses that include: a first concentrator region comprising: a first permeate channel that extends in a first serpentine pathway in a first layer, a first feed channel that extends in the first serpentine pathway in a second layer from a feed input to a first retentate output, wherein the first permeate channel extends adjacent to the first feed channel, and a first membrane region that separates the first permeate channel from the first feed channel; a dilution buffer region in fluid communication with the first retentate output of the first feed channel; a dilution buffer input into the dilution buffer region; and a second concentrator region comprising: a second permeate channel that extends in a second serpentine pathway in the first layer, a second feed channel in fluid communication with an output of the dilution buffer region, wherein the second feed channel extends in the second serpentine pathway in the second layer, wherein the second permeate channel extends adjacent to the second feed channel, and a second membrane region that separates the first permeate channel from the feed channel. The apparatus may include a second retentate output in fluid communication with the second feed channel.

The cross-sectional area of first feed channel may decrease along the first serpentine pathway from the feed input to the first retentate output to maintain a constant sheer rate within the first feed channel. For example, the height of the first feed channel may shorten along the first serpentine pathway from the feed input to the first retentate output. In some examples, the height of the first feed channel shortens along the first serpentine pathway from the feed input to the first retentate output and a height of the second feed channel shortens along the second serpentine pathway from the output of the dilution buffer region to the second retentate output.

In some examples, the first membrane region and the second membrane region may be part of a single membrane. In some examples, the first membrane region and the second membrane region are different membranes.

Any of these apparatuses may include a sealing structure comprising a pressure-distributing seal that distributes pressure to seal the first membrane region and the second membrane region between the first layer and the second layer. For example, the pressure-distributing seal may include a sheet of compressive foam. In some examples the pressure-distributing seal comprises a pressurizing chamber.

The first serpentine pathway may have a length of greater than about 5 meters. In some examples, the first membrane region has an area of greater than about 50 cm². The first membrane region and the second membrane region may comprise a membrane comprising an organic material. For example, the membrane may be a Polyethersulfone (PES) and/or a Composite Regenerated Cellulose (CRC) membrane. In some examples the first membrane region and the second membrane region have a high permeability for ethanol.

Any of these apparatuses may include a feed input port fluidly connected with the feed input and a retentate output port fluidly connected with the second retentate output. In some examples the apparatus includes a permeate output out of the first permeate channel at approximately the position along the overall path length of the dilution buffer input.

For example, described herein are apparatuses comprising: a first concentrator region comprising: a first permeate channel that extends in a first serpentine pathway in a first layer, a first feed channel that extends in the first serpentine pathway in a second layer from a feed input to a first retentate output, wherein the first permeate channel extends adjacent to the first feed channel, and a first membrane region that separates the first permeate channel from the first feed channel; a dilution buffer region in fluid communication with the first retentate output of the first feed channel; and a dilution buffer input into the dilution buffer region; and a second concentrator region comprising: a second permeate channel that extends in a second serpentine pathway in the first layer, a second feed channel in fluid communication with an output of the dilution buffer region, wherein the second feed channel extends in the second serpentine pathway in the second layer, wherein the second permeate channel extends adjacent to the second feed channel, and a second membrane region that separates the first permeate channel from the second feed channel; and a second retentate output in fluid communication with the second feed channel.

A cross-sectional area of first feed channel may decrease along the first serpentine pathway from the feed input to the first retentate output to maintain a constant sheer rate within the first feed channel. A height of the first feed channel may shorten along the first serpentine pathway from the feed input to the first retentate output. For example, a height of the first feed channel may shortens along the first serpentine pathway from the feed input to the first retentate output and wherein a height of the second feed channel shortens along the second serpentine pathway from an output of the dilution buffer region to the second retentate output. The first membrane region and the second membrane region may be parts of a single membrane. The apparatuses may include a pressure-distributing seal that distributes pressure to seal the first membrane region and the second membrane region between the first layer and the second layer.

The pressure-distributing seal may comprise a sheet of compressive foam. The pressure-distributing seal may comprise a pressurizing chamber. The first serpentine pathway may have a length of greater than about 5 meters. The first membrane region may have an area of greater than about 50 cm². The first membrane region and the second membrane region may each comprise a Polyethersulfone (PES), a Composite Regenerated Cellulose (CRC) membrane, or a combination thereof.

The first membrane region and the second membrane region may be permeable to ethanol. The apparatus further comprises a feed input port fluidly connected with the feed input and a retentate output port fluidly connected with the second retentate output. The apparatus may further include a permeate output out of the first permeate channel at approximately the dilution buffer input.

Also described herein are methods of concentrating and exchanging buffer of a solution using the microfluidic apparatuses described herein. For example, any of these methods may be methods of concentrating and exchanging buffer of a therapeutic polynucleotide solution in a microfluidic apparatus. These methods may include: passing the therapeutic polynucleotide solution through a first feed channel having a first serpentine length in a first region of the apparatus; filtering small molecules out of the therapeutic polynucleotide solution out of the first feed channel by tangential flow filtration into a permeate channel adjacent to the first feed channel through a first membrane region while maintaining a constant sheer rate relative to the first membrane region as the therapeutic polynucleotide solution passes through the first serpentine length of the first feed channel; and adding a buffer solution into the therapeutic polynucleotide solution after the first feed channel and concentrating the therapeutic polynucleotide solution in a second region of the apparatus.

Any of these methods may include maintaining a flow rate of the therapeutic polynucleotide solution in the first feed channel at a target flow rate. For example, any of these methods may include passing the therapeutic polynucleotide solution through the feed channel at a constant sheer rate. For example, passing may include passing at a constant sheer rate as a cross-sectional area of the first feed channel decreases along the first serpentine length. In some examples passing includes passing at a constant sheer rate as a height of the first feed channel decreases along the first serpentine length.

In general, these apparatuses may be configured so that the apparatuses increase the residence time in the single-pass tangential flow filtration region(s) to provide a concentrator factor of up to about 5 times (“5×”) or more (e.g., up to about 10 times or more, up to about 15 times or more, up to about 16 times or more, up to about 17 times or more, up to about 18 times or more, up to about 19 times or more, up to about 20 times or more, up to about 21 times or more, up to about 22 times or more, up to about 25 times or more, or higher.). For comparison, conventional multi-pass tangential flow filtration apparatuses have a concentration factor of 1.3× per pass. For example, these apparatuses may be configured to have a reduced flow rate of, e.g., about 4 ml/min or less, about 3 mL/min or less, about 2 mL/min or less, or lower. The flow rate of the therapeutic polynucleotide solution through the feed channel may be, e.g., between about 0.1 mL/min and 4 mL/min (e.g., between about 0.5 mL/min and 3 mL/min, between about 1 mL/min and 2 m/min, etc.). The actual flow rate may be determined specific to a particular application and/or apparatus configuration, since the flow rate may be affected by the optimum shear rate, initial concentration, target concentration factor, membrane permeability, maximum pressure, etc., or any combination of the foregoing.

The overall path length for the microfluidic concentrator and buffer exchange apparatus may be greater than about 5 meters (e.g., greater than about 6 meters, greater than about 7 meters, greater than about 8 meters, or higher). This overall path length may refer to the length of the feed channel through both the concentrator and the buffer exchange regions of the apparatus. The membrane area of the first membrane may be, e.g., between about 50-100 cm² (e.g., between about 60-80 cm², about 60 cm² or greater, about 70 cm² or greater, etc.). The apparatus may also maintain a target pressure at the input of the feed channel, e.g., between about 70 kPa and about 340 kPa (e.g., about 10 and about 50 psig), e.g., between about 100 kPa and 300 kPa, between about 150 kPa and about 250 kPa, etc.

In any of these methods, passing the therapeutic polynucleotide solution through the first feed channel may include passing the therapeutic polynucleotide solution along about 6 meters or more of the first feed channel.

For example, a method may comprise: passing a therapeutic polynucleotide solution through a first feed channel having a first serpentine length in a first region of a microfluidic apparatus; filtering small molecules out of the therapeutic polynucleotide solution out of the first feed channel by tangential flow filtration into a permeate channel adjacent to the first feed channel through a first membrane region while maintaining a constant sheer rate relative to the first membrane region as the therapeutic polynucleotide solution passes through the first serpentine length of the first feed channel; and adding a buffer solution into the therapeutic polynucleotide solution after the first feed channel and concentrating the therapeutic polynucleotide solution in a second region of the apparatus.

Any of these methods may include maintaining a flow rate of the therapeutic polynucleotide solution in the first feed channel at a target flow rate. Passing may include passing at a constant sheer rate. For example, passing may include passing at a constant sheer rate as a cross-sectional area of the first feed channel decreases along the first serpentine length. In some examples, passing may include passing at a constant sheer rate as a height of the first feed channel decreases along the first serpentine length.

Any of these methods may include maintaining a flow rate of the therapeutic polynucleotide solution in the first feed channel at a target flow rate, wherein the target flow rate is about 4 mL/min or less. Any of these methods may include maintaining a pressure in the first feed channel of between about 100 kPa and about 300 kPa. Passing the therapeutic polynucleotide solution through the first feed channel may include passing the therapeutic polynucleotide solution along about 6 meters or more of the first feed channel.

Some additional examples are provided below:

As mentioned above, these apparatuses may include an integrated concentrator and buffer exchange portion. These apparatuses may include a first concentrator-only region, such as those described above that is continuous and integrated with a second concentrator and buffer exchange region. For example, described herein are microfluidic concentrators and buffer exchange apparatuses that include: a permeate channel that extends in an elongate serpentine pathway in a first layer, a feed channel that extends in an elongate serpentine pathway in a second layer, wherein the permeate channel extends adjacent to a first length of the feed channel (along the length of the feed channel), a buffer channel that extends in an elongate serpentine pathway in a third layer, wherein the buffer channel extends adjacent to a second length of the feed channel that is a subsection of the first length of the feed channel and extends from a buffer input to a buffer output, a first membrane that separates the permeate channel from the feed channel and a second membrane that separates the buffer channel from the feed channel. These apparatuses may also include one or more flow sensors arranged to measure flow in the feed channel a start of the first length of the feed channel, at a start of the second length of the feed channel and at an end of the second length of the feed channel, and one or more valves (e.g., in the feed channel to regulate a flow rate in the feed channel). The first length of the feed channel may correspond to the concentrator-only region and the second length of the feed channel may be the integrated concentrator and buffer exchange region (which may be referred to herein as simply the buffer exchange region). The integrated concentrator and buffer exchange region may be configured so that the concentrator region is unitary with the buffer exchange region, forming a single module through which the feed channel extends, uninterrupted.

In some examples, these apparatuses (including the integrated concentrator and buffer exchange regions) may also include a sealing structure comprising a pressure-distributing seal adjacent to the first layer or the third layer that distributes pressure to seal the first membrane between the first layer and the second layer and the second membrane between the second layer and the third layer. The pressure-distributing seal may include a sheet of compressive foam, and/or pressurizing chamber that uses fluid pressure (e.g., air pressure) to distribute pressure across the apparatus to seal the membranes between their respective channels.

In any of these apparatuses, the feed channel, permeate channel and buffer channel may all be serpentine, so that the length of the channel may be many times greater than their diameter and height, which may be compactly arranged over the microfluidic apparatus. The feed channel, permeate channel and buffer channel may all be stacked atop each other. In general, the feed channel extends concurrently with the permeate channel over their entire length, although the permeate channel may include one or more intermediate outputs before the end of the permeate channel. However, the buffer channel in most of the examples described herein extends along only a portion of the length of the permeate and feed channels, such as over the last about 40% or less (e.g., last 35% or less, last 30% or less, last 25% or less, last 20% or less, or smaller) of the feed and/or permeate channels, depending on the desired reduction level of the specific component in the original solvent (e.g., ethanol). This configuration may allow a significant amount of concentration and buffer exchange to occur in line, with flow continuously proceeding through the integrated microfluidic concentrator and buffer exchange apparatus. For example, the buffer channel may take 15% or more of the total length of the feed channel to reduce the ethanol concentration by 5× (30% or more for 100× reduction, 50% or more for 1000× reduction, etc.).

As mentioned, the apparatuses described herein, including the apparatuses with integrated concentrator and buffer exchange modules may be configured so that the integrated concentrator and buffer exchange module increases the residence time in the single-pass tangential flow filtration pathway of the integrated concentrator and buffer exchange module to provide up to about 5 times or more (e.g., up to about 10 times or more, up to about 15 times or more up to about 16 times or more, up to about 17 times or more, up to about 18 times or more, up to about 19 times or more, up to about 20 times or more, up to about 21 times or more, up to about 22 times or more, up to about 25 times or more, or higher). For comparison, conventional multi-pass tangential flow filtration apparatuses have a concentration factor of 1.3× per pass. For example, these apparatuses may be configured to have a reduced flow rate of, e.g., about 4 ml/min or less (e.g., about 3 mL/min or less, about 2 mL/min or less, etc.). The flow rate of the therapeutic polynucleotide solution through the feed channel may be, e.g., between about 0.1 mL/min and 4 mL/min (e.g., between about 0.5 mL/min and 3 mL/min, between about 1 mL/min and 2 mL/min, etc.). The overall path length for the microfluidic concentrator and buffer exchange apparatus may be greater than about 5 meters (e.g., greater than about 6 meters, greater than about 7 meters, greater than about 8 meters, etc.). This overall path length may refer to the length of the feed channel through the integrated concentrator and buffer exchange apparatus. The membrane area of the first membrane may be, e.g., between about 50-100 cm² (e.g., between about 60-80 cm², about 60 cm² or greater, about 70 cm² or greater, etc.). The apparatus may also maintain a target pressure at the input of the feed channel, e.g., between about 70 kPa and about 340 kPa (e.g., about 10 and about 50 psig), e.g., between about 100 kPa and 300 kPa, between about 150 kPa and about 250 kPa, etc.

Any appropriate membrane may be used for the first and second membranes. The first and second membranes may be the same membrane material or may be different membrane materials. The first and second membranes may be relatively impermeable to the therapeutic polynucleotide (e.g., impermeable to the mRNA encapsulated in delivery vehicle). For example, the first and second membranes may comprise a membrane comprising an organic material. For example, the first or second membraned may be Polyethersulfone (PES) and/or a Composite Regenerated Cellulose (CRC) membrane. In some examples, the first membrane is a high-selectively membrane having a high permeability for solvent, such as ethanol and/or other solvents used in the synthesis of the therapeutic mRNA, and low binding to particles such as therapeutic mRNA nanoparticle comprising the mRNA and delivery vehicle. Example membranes may include Biomax™ Ultrafiltration Membrane 500 kDa (PES) (MilliporeSigma™, U.S.), Biomax™ Ultrafiltration Membrane, 50-500 kDa (PES) (MilliporeSigma™, U.S.), Ultracel™ Ultrafiltration Membrane, 30-300 kDa (CRC) (MilliporeSigma™, U.S.), ProStream™ Membrane, 30-300 kDa (PES) (Repligen™, U.S.), and Hystream™ Membrane, 30-300 kDa (PES) (Repligen™, U.S.).

The second membrane may have a lower permeability than the first membrane. For example, the second membrane, e.g., the exchange buffer membrane, may have a smaller permeability (flow rate/pressure) to solvent than the first membrane.

Any of these apparatuses may include a feed input port fluidly connected with the feed channel and retentate output port fluidly connected with an output of the feed channel. These apparatuses may include a second permeate output out of the permeate channel at approximately the buffer input.

In some examples, the first layer, the second layer and the third layer each include a microfluidic body having a buffer port in fluid communication with the buffer channel and a permeate port in fluid communication with the permeate channel. In any of these apparatuses the height of the feed channel may be constant over the first length of the feed channel. Alternatively, in some examples the height of the feed channel decreases over the first length of the feed channel.

In some examples the height of the buffer channel may decrease over the second length of the feed channel.

In some example, a microfluidic concentrator and buffer exchange apparatus includes: a permeate channel extending in an elongate serpentine pathway in a first layer; a feed channel extending in an elongate serpentine pathway in a second layer, wherein the permeate channel extends adjacent to a first length of the feed channel from a permeate input to a permeate output; a buffer channel extending in an elongate serpentine pathway in a third layer, wherein the buffer channel extends adjacent to a second length of the feed channel that is a subsection of the first length of the feed channel extending from a buffer input to a buffer output; a first membrane separating the permeate channel from the feed channel; a second membrane separating the buffer channel from the feed channel; one or more flow sensor arranged to measure flow in the feed channel a start of the first length of the feed channel, at a start of the second length of the feed channel and at an end of the second length of the feed channel; and a first valve at the permeate output; a second valve at the buffer output; and a pressure-distributing seal adjacent to the first layer or the third layer that distributes pressure to seal the first membrane between the first layer and the second layer and the second membranes between the second layer and the third layer.

Also described herein are method of concentrating and exchanging buffer of a therapeutic polynucleotide solution (e.g., a solution of a therapeutic mRNA encapsulated in a delivery vehicle) using any of the microfluidic apparatuses described herein. For example, a method may include: passing the therapeutic polynucleotide solution through a feed channel having a serpentine first length; filtering small molecules out of the therapeutic polynucleotide solution in the feed channel by tangential flow filtration through into a permeate channel adjacent to the feed channel through a first membrane separating the feed channel from the permeate channel as the therapeutic polynucleotide solution passes in the first length of the feed channel; introducing a buffer solution into the therapeutic polynucleotide solution in the feed channel from a buffer channel that is adjacent to the feed channel through a second membrane separating the feed channel from the buffer channel as the therapeutic polynucleotide solution passes in a second length of the feed channel that is a subsection of the first length of the feed channel; and maintaining a flow rate of the therapeutic polynucleotide solution in the feed channel at a target flow rate.

These methods may include increasing a concentration factor while passing the therapeutic polynucleotide solution through the feed channel by about 20-fold or more. These methods may include passing at a constant sheer rate. For example, passing may include passing at a constant sheer rate by decreasing a height of the feed channel over a portion of the first length before the second length of the feed channel.

Maintaining the flow rate at the target flow rate may include maintaining the target flow rate at about 4 mL/min or less (e.g., about 2 mL/min or less, about 1 m/min or less, etc.). The process of maintaining the flow rate at the target flow rate may comprise maintaining a pressure in the feed channel (e.g., in the input to the feed channel) of between about 100 kPa and 300 kPa (e.g., between about 150 kPa and about 250 kPa, etc.).

The process of passing the therapeutic polynucleotide solution through the feed channel may include passing the therapeutic polynucleotide solution along about 5 meters or more (e.g., about 6 meters or more, about 7 meters or more, about 8 meters or more, or higher) of the first length.

Any of these methods may include maintaining the flow rate of the therapeutic polynucleotide solution in the feed channel by increasing a channel resistance of the buffer channel over a region of the buffer channel adjacent to the second length of the feed channel. For example, maintaining the flow rate of the therapeutic polynucleotide solution in the feed channel may include increasing a channel resistance of the buffer channel over a region of the buffer channel adjacent to the second length of the feed channel by decreasing a height of the buffer channel.

Any of these microfluidic apparatuses may include one or more fluid pumps configured to pump fluid through the single pass concentrator and buffer exchange system. For example, the pump may be a syringe pump, a pressure pump, a peristaltic pump, etc. In some example, the pump may be a membrane pump that is part of the microfluidic apparatus; for example, the pump may be formed by deflecting at least a portion of an elastic membrane within the microfluidic apparatus. The microfluidic path apparatus may include one or more fluid pumps in front of the permeate channel, feed channel, and/or buffer channel(s). Alternatively or additionally, any of these apparatuses (e.g., any of these microfluidic apparatuses) described herein may use a non-pulsatile pressure source to drive fluid. Thus, the flow through the mixer may be continuous and non-pulsing.

The microfluidic concentrators and buffer exchange components described herein, as well as microfluidic apparatuses (e.g., microfluidic chips) that include them, may be extremely compact and efficient and may operate on or within the bounds of a microfluidic apparatus with high efficiency and accuracy.

Any of the apparatuses (e.g., microfluidic apparatuses) and methods described herein may involve tangentially flowing a therapeutic composition (e.g., a therapeutic polynucleotide, including therapeutic mRNA encapsulated in a delivery vehicle) through a feed channel, as described above. The feed channel may have an ultrafiltration membrane on either side (e.g., top and bottom) that limits (and in some cases prevents) the diffusion and/or infusion of nanoparticles but allows the diffusion of smaller particles, such as solvents and ions, to pass through the membrane as a permeate material, to waste, thereby concentrated therapeutic particles (e.g., mRNA encapsulated in delivery vehicle), while a new buffer may be applied through the membranes that separates the feed channel from the buffer channel. The concentrated therapeutic particles, in the new buffer solution, may be collected downstream as retentate. In some cases, a biocompatible and stable buffer can be used for downstream processing for injection to patients. Buffer adjustment may be accomplished by adding diluent with appropriate composition of water, salt, excipients, and/or other constituents. The concentration of certain chemicals may be increased by adding a buffer with higher chemical concentration and vice versa.

The methods and apparatuses described herein can allow for the formulation of the biomolecule-containing product, buffer adjustment and concentration to be performed in one microfluidic apparatus. Thus, the formulation buffer may be adjusted to a more biocompatible and stable buffer for downstream processing and injection to patients. The therapeutic concentration may be also adjusted to an acceptable volume for the therapeutic administration method after formulation and buffer adjustment process.

For example, described herein are microfluidic devices, the devices comprising: a permeate channel that extends in an elongate serpentine pathway in a first layer; a feed channel that extends adjacent to the permeate channel in a second layer; a buffer channel that extends, in a third layer, adjacent to a subsection of the a length of the feed channel from a buffer input to a buffer output; a first membrane that separates the permeate channel from the feed channel; a second membrane that separates the buffer channel from the feed channel; and wherein a height of the buffer channel decreases over the second length of the feed channel.

Any of these devices may include one or more flow sensors arranged to measure flow in the feed channel a start of the feed channel, at a start of the length of the feed channel adjacent to the buffer input, and at an end of the feed channel. Any of these devices may include one or more valves in the feed channel to regulate a flow rate in the feed channel.

In some examples, the devices described herein include a pressure-distributing seal adjacent to the first layer or the third layer that distributes pressure to seal the first membrane between the first layer and the second layer and the second membrane between the second layer and the third layer. The pressure-distributing seal may comprise a sheet of compressive foam. In some examples the pressure-distributing seal comprises a pressurizing chamber. The feed channel may have a length of greater than about 5 meters. The first membrane may have an area of greater than about 50 cm². The first and second membranes may comprise a Polyethersulfone (PES), a Composite Regenerated Cellulose (CRC) membrane, or a combination of these. The second membrane may have a lower permeability than the first membrane.

Any of these devices may include a feed input port fluidly connected with the feed channel and retentate output port fluidly connected with an output of the feed channel. Any of these devices may include a second permeate output out of the permeate channel at approximately the buffer input.

The first layer, the second layer and the third layer may include a microfluidic body having a buffer port in fluid communication with the buffer channel and a permeate port in fluid communication with the permeate channel. The height of the feed channel may be constant over the first length of the feed channel. In some examples, the height of the feed channel decreases over the first length of the feed channel.

For example, a microfluidic device may include: a permeate channel that extends in an elongate serpentine pathway in a first layer; a feed channel that extends adjacent to the permeate channel in a second layer; a buffer channel that extends, in a third layer, adjacent to a subsection of a length of the feed channel from a buffer input to a buffer output; a first membrane that separates the permeate channel from the feed channel; and a second membrane that separates the buffer channel from the feed channel; wherein a height of the buffer channel decreases over the second length of the feed channel.

Any of these devices may include one or more flow sensors arranged to measure flow in the feed channel a start of the feed channel, at a start of the length of the feed channel adjacent to the buffer input, and at an end of the feed channel.

In some examples, the device includes one or more valves in the feed channel to regulate a flow rate in the feed channel. The device may include a pressure-distributing seal adjacent to the first layer or the third layer that distributes pressure to seal the first membrane between the first layer and the second layer and the second membrane between the second layer and the third layer. The pressure-distributing seal may include a sheet of compressive foam. The pressure-distributing seal may include a pressurizing chamber. The feed channel may have a length of greater than about 5 meters. The first membrane may have an area of greater than about 50 cm².

Each of the first and second membranes may comprise a Polyethersulfone (PES), a Composite Regenerated Cellulose (CRC) membrane, or a combination of these. The second membrane may have a lower permeability than the first membrane.

Any of these devices may include a feed input port fluidly connected with the feed channel and retentate output port fluidly connected with an output of the feed channel. In some examples, the devices include a second permeate output out of the permeate channel at approximately the buffer input.

The first layer, the second layer and the third layer may include a microfluidic body having a buffer port in fluid communication with the buffer channel and a permeate port in fluid communication with the permeate channel. In some examples, a height of the feed channel may be constant over the first length of the feed channel. In some examples, a height of the feed channel decreases over the first length of the feed channel.

For example, a microfluidic device may include: a permeate channel extending in an elongate serpentine pathway in a first layer; a feed channel extending in a second layer, wherein the permeate channel extends adjacent to a first length of the feed channel from a permeate input to a permeate output; a buffer channel extending in a third layer, wherein the buffer channel extends adjacent to a second length of the feed channel that is a subsection of the first length of the feed channel extending from a buffer input to a buffer output; a first membrane separating the permeate channel from the feed channel; a second membrane separating the buffer channel from the feed channel; one or more flow sensors arranged to measure flow in the feed channel a start of the first length of the feed channel, at a start of the second length of the feed channel and at an end of the second length of the feed channel; a first valve at the permeate output; a second valve at the buffer output; and a pressure-distributing seal adjacent to the first layer or the third layer that distributes pressure to seal the first membrane between the first layer and the second layer and the second membranes between the second layer and the third layer.

Also described herein methods for concentrating and exchanging buffer, including: passing a therapeutic polynucleotide solution through a feed channel of a microfluidic device, the feed channel having a serpentine first length; filtering small molecules out of the therapeutic polynucleotide solution in the feed channel by tangential flow filtration through into a permeate channel adjacent to the feed channel through a first membrane separating the feed channel from the permeate channel as the therapeutic polynucleotide solution passes in the first length of the feed channel; introducing a buffer solution into the therapeutic polynucleotide solution in the feed channel from a buffer channel that is adjacent to the feed channel through a second membrane separating the feed channel from the buffer channel as the therapeutic polynucleotide solution passes in a second length of the feed channel that is a subsection of the first length of the feed channel; and maintaining a flow rate of the therapeutic polynucleotide solution in the feed channel at a target flow rate.

Any of these methods may include increasing a concentration factor while passing the therapeutic polynucleotide solution through the feed channel by about 20-fold or more. Passing may include passing at a constant sheer rate along a portion of the first length of the feed channel upstream of the second length. In some examples, passing comprises passing at a constant sheer rate along a portion of the first length of the feed channel upstream of the second length of the feed channel by decreasing a height of the feed channel over the portion of the first length upstream of the second length of the feed channel.

Maintaining the flow rate at the target flow rate may comprise maintaining the target flow rate in the feed channel at about 4 m/min or less over the first and length of the feed channel. Any of these methods may include maintaining a same flow rate of buffer solution from the buffer channel into the feed channel as the flow rate of small molecules in solution from the feed channel into the permeate channel over the second length of the feed channel. Maintaining the flow rate at the target flow rate may comprise maintaining a pressure in the feed channel over the first length of the feed channel of between about 100 kPa and 300 kPa.

In any of these methods, passing the therapeutic polynucleotide solution through the feed channel may comprise passing the therapeutic polynucleotide solution along about 6 meters or more of the first length. Maintaining the flow rate of the therapeutic polynucleotide solution in the feed channel may comprise increasing a channel resistance of the buffer channel over a region of the buffer channel adjacent to the second length of the feed channel. Maintaining the flow rate of the therapeutic polynucleotide solution in the feed channel may comprise increasing a channel resistance of the buffer channel over a region of the buffer channel adjacent to the second length of the feed channel by decreasing a height of the buffer channel.

All of the methods and apparatuses described herein, in any combination, are herein contemplated and can be used to achieve the benefits as described herein.

### BRIEF DESCRIPTION OF THE DRAWINGS

The novel features of the methods and apparatuses (e.g., devices) are set forth with particularity in the claims that follow. A better understanding of the features and advantages of these methods and apparatuses will be obtained by reference to the following detailed description that sets forth illustrative examples, in which the principles of the invention are utilized, and the accompanying drawings of which:

FIG. 1A schematically illustrates one example of a microfluidic apparatus for concentrating and exchanging buffer in a solution, as described herein. In FIG. 1A the apparatus includes serially arranged single-pass, tangential flow concentrator regions separated by a dilution region, to concentrate and replace the buffer in the feed solution. Optionally, additional dilution and concentration regions may be included to further concentrate and/or exchange buffer in the solution.

FIG. 1B shows another example of a microfluidic apparatus for concentrating and exchanging buffer in a solution. In FIG. 1B the apparatus includes a first single pass, tangential flow concentrator region serially connected with a dilution region and a second single pass, tangential flow concentrator region.

FIG. 1C schematically illustrates another example of a microfluidic apparatus for concentrating and exchanging buffer in a solution. In FIG. 1C, the height of the feed channel in the concentrator regions decreases along the length of the feed channel.

FIG. 2A shows one example of a microfluidic apparatus including a single pass, tangential flow concentrator region as described herein.

FIG. 2B shows an enlarged view of the single pass, tangential flow concentrator (concentrator region) of the microfluidic apparatus of FIG. 2A.

FIG. 2C is a section view through a portion of the single pass, tangential flow concentrator region of FIG. 2B.

FIGS. 3A-3C illustrate sealing structures for a single pass, tangential flow concentrator (concentrator region) in which one (or more) membranes are secured between the layers forming the feed channel and permeate channel.

FIG. 4A schematically illustrates an example of a microfluidic apparatus for concentrating and exchanging buffer in a solution. In FIG. 4A the example apparatus including a single pass, tangential flow concentrator integrated with a buffer exchange region.

FIG. 4B schematically illustrates another example of a microfluidic apparatus for concentrating and exchanging buffer in a solution. In FIG. 4B a single pass, tangential flow concentrator region is integrated with a buffer exchange region in which the height of the feed channel in the concentrator region decreases along a portion of the length of the feed channel.

FIG. 4C schematically illustrates another example of a microfluidic apparatus for concentrating and exchanging buffer in a solution. In FIG. 4C a single pass, tangential flow concentrator region is integrated with a buffer exchange region in which the height of the feed channel decreases along a portion of the length of the feed channel in the concentrator region and the height of the buffer channel decreases along its length in the buffer exchange region.

FIG. 5A schematically illustrates one example of a buffer exchange region of a microfluidic apparatus in which the buffer exchange region is integrated with a single pass, tangential flow concentrator as described herein.

FIG. 5B schematically illustrates another example of a buffer exchange region of a microfluidic apparatus in which the buffer exchange region is integrated with a single pass, tangential flow concentrator. In FIG. 5B the channel height of the buffer channel is decreased to increase channel resistance within the buffer channel.

FIGS. 6A-6B schematically illustrate one example of a microfluidic apparatus for concentrating and exchanging buffer in a solution including a single pass, tangential flow concentrator region integrated with a buffer exchange region. FIG. 6A shows a microfluidic single pass, tangential flow concentrator including a buffer exchange region, while FIG. 6B shows a section through a portion of the single pass, tangential flow concentrator and buffer exchange region of FIG. 6A.

FIG. 7 schematically illustrates one example of a microfluidic apparatus for concentrating and exchanging buffer in a solution including a single pass, tangential flow concentrator and a buffer exchange region.

FIG. 8 schematically illustrates one example of a microfluidic apparatus including a concentrator showing an arrangement of valve and flow sensors.

FIG. 9 schematically illustrates one example of a plurality of cascaded single-pass tangential flow filtration modules (e.g., concentrators) that may achieve higher flow rates using smaller path lengths.

FIG. 10 schematically illustrates one example of a microfluidic apparatus for concentrating and exchanging buffer in a solution similar to that shown in FIG. 1A.

FIG. 11 schematically illustrates one example of a microfluidic apparatus in which mixing, and dilution of buffer is done off of the microfluidic chip.

FIG. 12 schematically illustrates one example of a microfluidic apparatus including a concentrator and buffer exchange region.

FIG. 13 schematically illustrates one example of a microfluidic apparatus including a concentrator and buffer exchange region including off-chip storage.

### DETAILED DESCRIPTION

In general, described herein are apparatuses (e.g., systems, devices, etc.) and methods for concentrating and exchanging buffer for solutions of biomolecules, such as therapeutic polynucleotides including, but not limited to, therapeutic mRNAs. For example, these apparatuses and methods may reduce, minimize, or in some instances even eliminate, manual handling during formulation of therapeutic mRNAs encapsulated in delivery vehicle. The apparatuses and methods described herein may have a benefit of providing concentration by a factor of twenty-fold and exchange of buffer in a compact microfluidic environment that may be aseptic and may provide a sterile path for processing a final therapeutic composition that may be stored and/or directly applied to a patient.

The methods and apparatuses described herein may generate therapeutics at relatively rapid cycle times at relatively high degree of reproducibility. The apparatuses described herein may be part of a single integrated apparatus that performs synthesis, purification, compounding, and concentration of one or more therapeutic composition (including, but not limited to therapeutic polynucleotides). All or some of these processes may be performed in an unbroken fluid processing pathway, which may be configured as one or a series of consumable microfluidic apparatus(es), which may also be referred to as a microfluidic path chip, microfluidic path plate, process chip, biochip, or process plate. This may allow for patient-specific therapeutics to be synthesized, including compounding, at a point of care (e.g. hospital, clinic, pharmacy, etc.).

During operation of the apparatus the fluid path may remain substantially unbroken, and contamination may be substantially reduced (or in some cases eliminated) by non-contact monitoring (e.g., optically monitoring), including fluid flow measurement, mixing monitoring, etc. and by manipulating precise microfluidic amounts (metering, mixing, etc.) using pressure applied from a deflectable membrane on an opposite side of the fluidic chambers and channels. As used herein, a substantially unbroken fluid path means a continuous fluid path that is not interrupted by one or more openings, such as openings exposing fluid within the fluid path to air.

These apparatuses and methods may be configured for use at a point of care. For example, the methods and apparatuses described herein may be configured for manufacturing customized therapeutic compositions including one or more therapeutic polynucleotide (e.g., mRNA, microRNA, DNA, etc.).

The methods and apparatuses described herein may provide scalable polynucleotide manufacturing, production of single patient dosages, reduction (and in some instances even elimination) of touchpoints to limit contamination, input and process tracking for meeting clinical manufacturing requirements, and use in point-of-care operations for therapeutics.

The microfluidic concentrators and buffer exchange apparatuses described herein may be included as part of a microfluidic path apparatus (e.g., microfluidic path device). The microfluidic path apparatus may be configured as a microfluidic chip or a microfluidic cartridge. A microfluidic cartridge may include a microfluidic chip. For example, any of these microfluidic concentrators and buffer exchange apparatuses may be formed as part of a microfluidic chip or cartridge and may include an input and output port for the buffer to be exchanged and the therapeutic solution being processed, and an output for the permeate solution. Any appropriate therapeutic solution may be processed by the apparatuses and methods described herein. These solutions may include a biomolecule that will be retained in the therapeutic solution so that it may be concentrated. The biomolecule is generally sufficiently large so that it is retained. For example, the biomolecule may be a therapeutic polynucleotide such as a therapeutic mRNA; the biomolecule may be a therapeutic mRNA that is combined with (e.g., fully or partially encapsulated by) a delivery vehicle. The therapeutic solution may be referred to as a feed input or simply the feed. The output for the therapeutic solution that is retained following concentration and/or buffer exchange may be referred to as the retentate solution of retentate.

The microfluidic concentrator and buffer exchange apparatuses described herein both concentrate and remove an unwanted material from the therapeutic solution as part of a single-pass tangential flow filtration process in which a first ultrafiltration membrane is used to separate therapeutic nanoparticles from the solvents. Extra solvents, which are permeable to the first ultrafiltration membrane will pass through the first membrane to waste (as permeate), thereby concentrating the therapeutic nanoparticles in the same solution. Concentration may be performed in an upstream portion of the apparatus, while the downstream portion may also include buffer exchange by dilution (e.g., adding new buffer and concentrating again).

The microfluidic concentrator and buffer exchange apparatuses described herein may therefore initially concentrate the therapeutic solution through a concentrator portion, which may be referred to as a first region, that this upstream including a feed channel holding the therapeutic solution that is separated by a permeate channel receiving the permeate solution; partway through the apparatus a buffer exchange region, which may be referred to as a second region, may be coupled in-line with the first concentrator portion. In some examples the buffer exchange region may include a dilution region after the concentrator, in which additional new buffer is added to the retentate that was concentrated to dilute out the previous buffer; the diluted retentate may then be concentrated again, using another (or the same) concentrator.

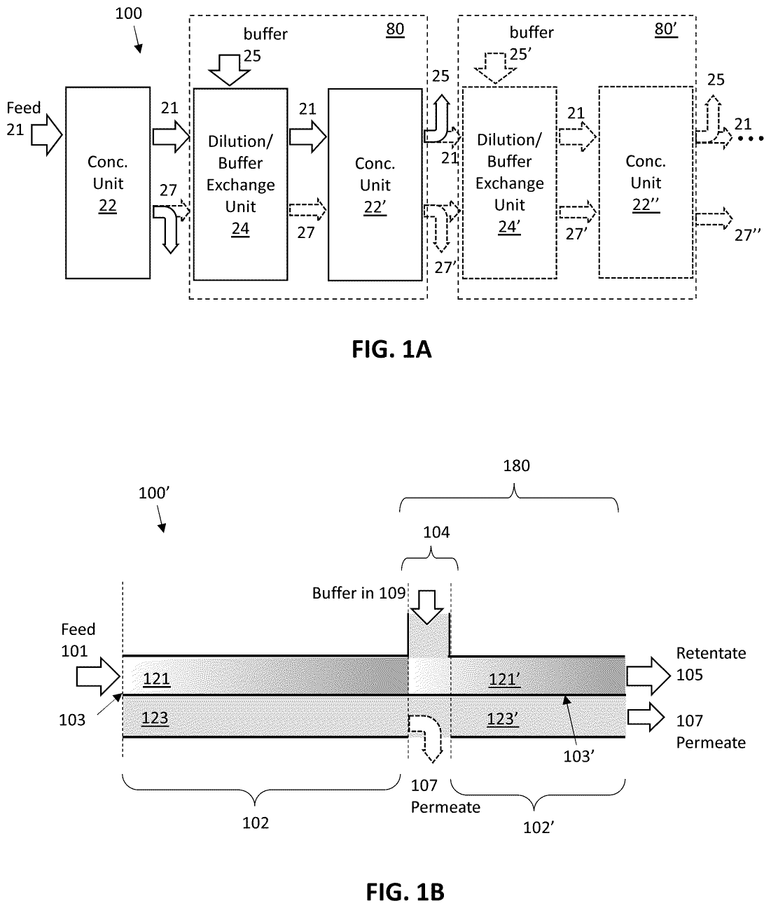

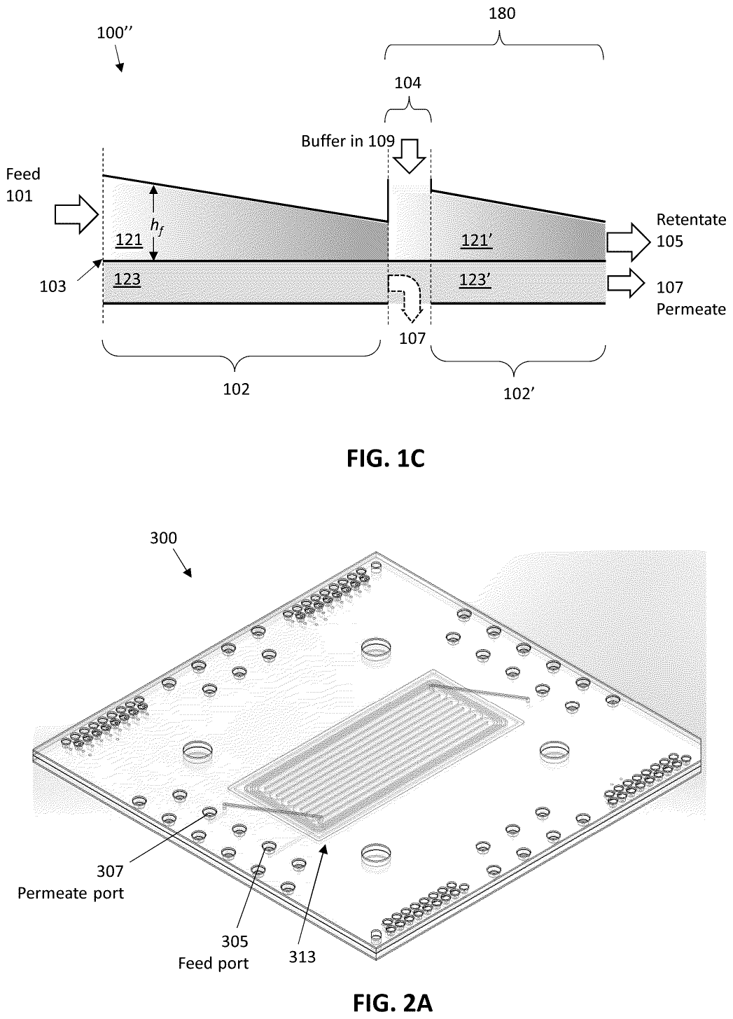

For example, FIG. 1A is a schematic illustrating one example of an apparatus for concentrating and exchanging buffer in a solution. In FIG. 1A, the apparatus includes a feed input, into which the feed solution 21 is added to the apparatus (e.g., system 100). The feed solution is initially input into a concentration unit 22. The concentration unit (also referred to herein as a concentration region of the apparatus), may include a first permeate channel that extends in a first serpentine pathway in a first layer, a first feed channel that extends in the first serpentine pathway in a second layer from a feed input to a first retentate output, wherein the first permeate channel extends adjacent to the first feed channel, and a first membrane region that separates the first permeate channel from the first feed channel. Examples of concentration regions are described in further detail below. The concentration unit may be directly (in series, in-line, etc.) coupled to a dilution and/or buffer exchange unit or region 24. In some examples the dilution region may be a chamber or channel fluidly connected with the retentate output of the concentrator into which new buffer 25, 25′ may be added. In some examples the new buffer is added directly into the feed channel holding the retentate. The apparatus may then include a second concentrator region 22′ which may once again concentrate the feed (the diluted retentate) to form a second retentate 21′. In FIG. 1A, the dilution and concentrator module 80 includes both a dilution/buffer exchange unit 24 and an additional concentrator unit 22′. The permeate from the first concentrator unit 22 may exit the apparatus or it may be passed through to the second concentrator unit 22′ through the dilution/buffer exchange unit 24. One or more additional (e.g., two or more, three or more, four or more, five or more, etc.) sequential dilution and concentrator modules 80′ may be connected in series with the first concentrator 22 and/or the first dilution and concentrator module 80. The feed solution (retentate) 21 from each module may then passed on to the next module. Thus, as shown in FIG. 1A in some examples the microfluidic apparatus may include sequentially arranged concentrators and buffer exchange modules; each buffer exchange module may include a dilution/buffer exchange unit and a concentration unit. In another example, additional dilution and concentration regions may be included to further concentrate and/or exchange buffer in the solution.

FIG. 1B illustrates another example of an apparatus 100′ that includes a first concentrator region 102 that is fluidly coupled in series with a dilution and concentration module 180 including a dilution region 104 and second concentration region 102′. In this example the feed solution 101 (such as a therapeutic solution) is input into the feed channel 121 of the first concentrator region 102. The first concentrator region also includes a first permeate channel 123. Both the first feed channel and the first permeate channel may be serpentine channels (not apparent in the section view shown in FIG. 1B), that extend adjacent to each other and are separated by a membrane 103 through which smaller molecules may pass as the feed solution is driven through first feed channel while the flow is maintained at a constant sheer rate of the feed solution relative to the membrane. The permeate may flow in the same direction and may exit the first concentration region 102 at a permeate output 107 and/or may pass into the second concentration region 102′ of the dilution and concentration module 180, e.g., passing through the dilution region 104. The dilution region includes an input for dilution buffer 109 that may be in direct fluid communication with the retentate from the feed channel. The diluted retentate may then be passed into the second concentrator region 102′ which may, like the first concentrator region 102, include a permeate channel (second permeate channel 123′) and a feed channel (second feed channel 121′) separated by a membrane region 103′. The second membrane region may be part of the same membrane as the first membrane region or it may be part of a second membrane. The retentate 105 that is output from the second concentrator region 102′ may then be passed on to a second (or more) dilution and concentration module (as shown in FIG. 1A) or it may be output.

Any of the apparatuses described herein may be configured so that the concentration region(s) are adapted to maintain a constant sheer rate within the feed channel along the length of the feed channel, which may improve the single-pass, single flow filtration, allowing very high concentration ratios. As used herein a constant sheer rate refers to a sheer rate that is approximately constant along the length of the feed channel, for example, rates that vary by about 10% in magnitude or less (e.g., about 9% or less, about 8% or less, about 7% or less, about 6% or less, about 5% or less, about 4% or less, about 3% or less, about 2% or less, about 1% or less, or smaller.).

The apparatus may be configured to achieve a constant sheer rate by controlling the cross-sectional area of the feed channel. For example, the feed channel may generally decrease the cross-sectional area along the length of the feed channel adjacent to the permeate channel (separated by the membrane). In some examples the width of the portion of the feed channel contacting the membrane opposite from the permeate channel may be constant and the height may decrease along the length, as shown in FIG. 1C, which may maintain a constant sheer rate along the length of the feed channel 121.

In FIG. 1C the apparatus 100″ includes a first concentrator region 102 that is similar to that shown in FIG. 1B, but the height of the feed channel, h_{f}, decreases along the length of the feed channel 121, in some examples the serpentine pathway that the feed channel travels. The width of the feed channel (not visible in FIG. 1C) may be constant, and the cross-sectional area of the permeate channel 123 may also remain constant. In some examples, as shown in FIG. 1C, the second concentrator region 102′ also includes a height of the feed channel that decreases along the length of the second feed channel 121′. The change in the cross-sectional area along the length (e.g., the change in the height) may be linear or non-linear; in some examples the change in cross-sectional area (e.g., height) may increase along the length; in other examples the change in cross-sectional area (e.g., height) may decrease along the length.

In FIGS. 1A-1C the permeate solution may flow in the permeate channel in the same direction as the therapeutic solution in the feed channel.

In use, any of the microfluidic concentrator and buffer exchange apparatuses, including examples with multiple concentrators and dilution/buffer exchange modules arranged in series as well as integrated concentrator and buffer exchange modules as described below, may be highly efficient and may concentrate a manufactured dose of therapeutic solution into a concentration range that allows dilution to an injectable dose form (e.g., between 2 mL and 0.1 mL).

As mentioned, the microfluidic concentrator and buffer exchange apparatuses described herein are single-pass concentrators that operate using a modified tangential flow filtration. In some examples buffer exchange and concentration may be performed as a single pass step with upstream concentration.

An unfiltered therapeutic solution (“feed solution”) is fed into a feed inlet and passed through the feed channel under pressure. The first membrane allows a permeate, such as small molecules (e.g., ethanol and/or some salts), to pass through the first membrane and into the permeate channel as the feed solution passes tangentially over the membrane. The permeate flow through the permeate channel to exit the permeate channel via one or more permeate channel outlets. The first membrane may hinder (and in some instances even prevent) the retentate, which includes the therapeutic particles, from passing through the first membrane. The flow of feed solution through the feed channel allows the retentate to exit the feed channel via one or more retentate outlets. The concentration of the therapeutic particles (e.g., therapeutic mRNA with delivery vehicle) generally increases as the therapeutic mixture passes through the feed channel, providing a higher concentration of the therapeutic material at the retentate outlet. Fluid flow along the first membrane creates a shear force to remove particles from the first membrane to reduce (and in some even prevent) membrane clogging and enables large-scale processing compared to dead-end filtration.

The concentration factor can be proportional to the feed flow rate relative to the retentate flow rate (feed flow rate/retentate flow rate). The retentate flow rate relative to the permeate flow rate (retentate flow rate/permeate flow rate) can be controlled by restricting the flow of retentate. In general, higher retentate flow resistance is associated with higher transmembrane pressure, higher transmembrane flow rate, higher permeate/retentate flow rate ratio, and a higher concentration factor.

The microfluidic concentrator and buffer exchange apparatuses described herein may increase the concentration factor during the single pass through the apparatus by increasing the residence time in the apparatus. The apparatuses (including any suitable systems and devices) described herein may achieve a target concentration factor of about five-fold or greater—e.g., about 10-fold or greater, about 15-fold or greater, about 20-fold or greater, about 25-fold or greater, or higher. In one example, the concentration factor is about 20-fold or greater. These aforementioned concentration factors are particularly surprising given the concentration factor of conventional multi-pass tangential flow systems is 1.3-fold per pass. The remarkably high concentration factors may be due to one or more (or the combination of two or more) of the following: the geometry and/or arrangement of the permeate, feed, and buffer channels, the control of flow in the retentate channel, the buffer channel and/or the feed channel (in variations including a feed channel), the length and diameter (e.g., volume) of the feed channel (in variations including a feed channel), retentate channel and/or the buffer channel, the membrane area, and/or the pressure in the feed channel (in variations including a feed channel), and retentate channel and/or the buffer channel.

For example, the microfluidic concentrator and buffer exchange apparatuses described herein may have a relatively low flow rate and a relatively long fluid path. The flow rate may be about 4 mL/min or less (e.g., about 3 m/min or less, about 2 mL/min or less, about 1 mL/min or less, or smaller.). In one example, the feed and retentate channels may be relatively long and relatively narrow, and arranged in a relatively compact, serpentine path.

The microfluidic concentrator and buffer exchange apparatuses described herein may regulate the flow rate within the feed channel, permeate channel and/or the buffer channel. Flow rate in one or more of these channels may be monitored by one or more flow sensors. The flow sensors may be integrated into the apparatus. For example, a flow sensor may be configured to measure flow in the feed channel at feed port; a second flow sensor may be configured to measure flow at the outlet (e.g., the retentate outlet) of the feed channel. In some examples flow sensors for measuring flow within the feed channel may be part of the second layer. Flow sensors may also be configured to measure flow into the permeate channel, and/or out of the permeate channel. Each of these flow sensors may be part of the layer including the channel. Flow sensors may also measure flow through one or more intermediate regions of the feed channel, and/or permeate channel.

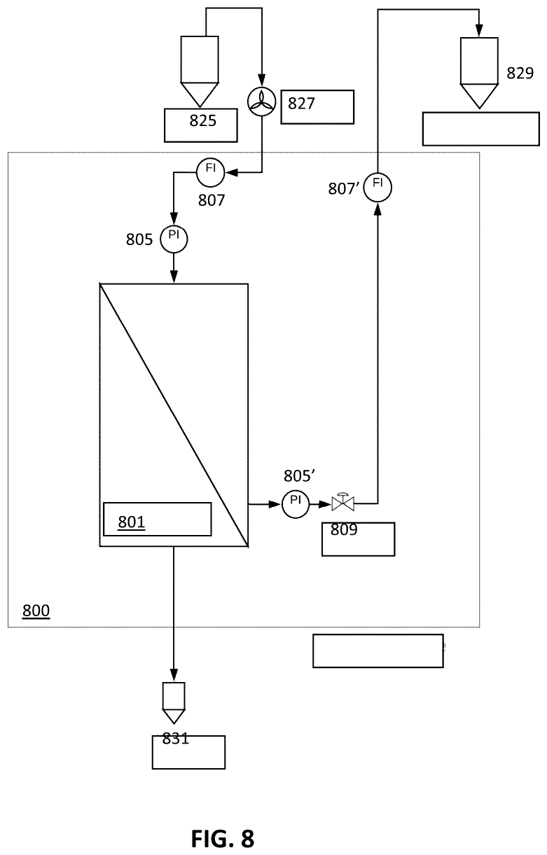

For example, FIG. 8 shows a schematic of an example of a concentrator device 800 in which multiple flow rate sensors 807, 807′ are included with pressure sensors 805, 805′ and a valve 809. In FIG. 8, the pressure indicator/sensor 805 is positioned at the start of the feed into the concentration module 801. The concentrator device 800 includes a pair of flow sensors 807, 807′ that are positioned at the start (feed input) and end (feed output or retentate output) of the concentrator on the device (“chip”). The control valve 809 at the exit of the concentrator module may be used to regulate the pressure and flow based on feedback from the sensors. The device shown in FIG. 8 also includes a pump 827 for pumping input material (e.g., raw drug product 825) through the concentration module, with permeate leaving as waste 831 and retentate leaving as concentrated therapeutic 829.

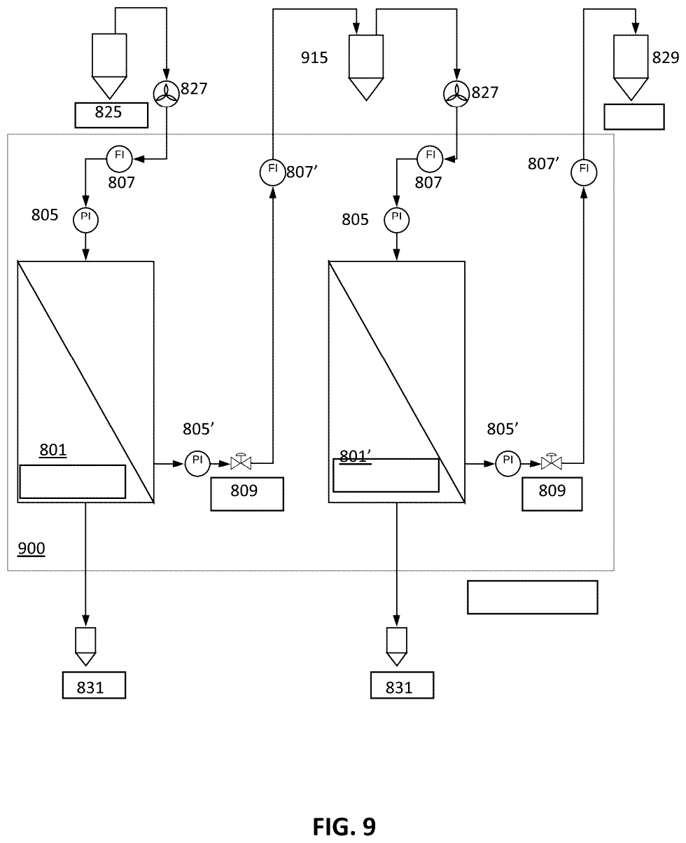

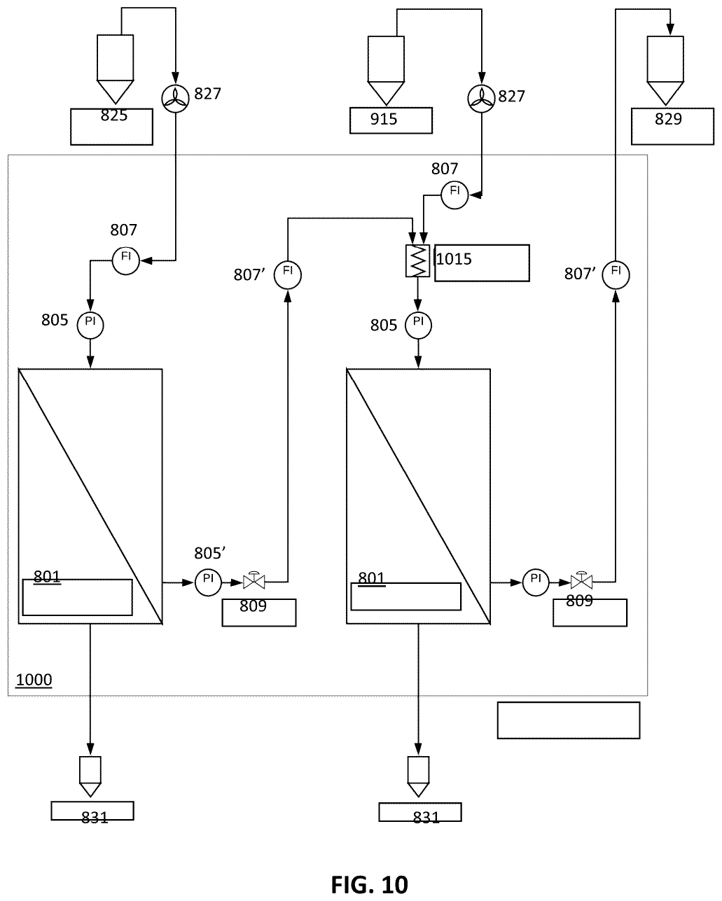

The example concentrator and buffer exchange microfluidic device 900 shown schematically in FIG. 9 includes pressure sensors (PI) and flow sensors (F1) at the inputs and outputs of each feed pathway of the (tangential flow filtration) concentration modules 801, 801′ that are connected in tandem. Control valves after each concentrator module may be used to regulate pressure and/or flow. An external (e.g., off of the device) input 915 may be used to add new buffer solution following the first concentrator, in order to dilute out the original buffer. The device 1000 of FIG. 10 is similar to that shown in FIG. 9 but includes a mixer 1015 for receiving dilution buffer 915 and mixing it with the concentrated retentate from the first stage concentrator 801.

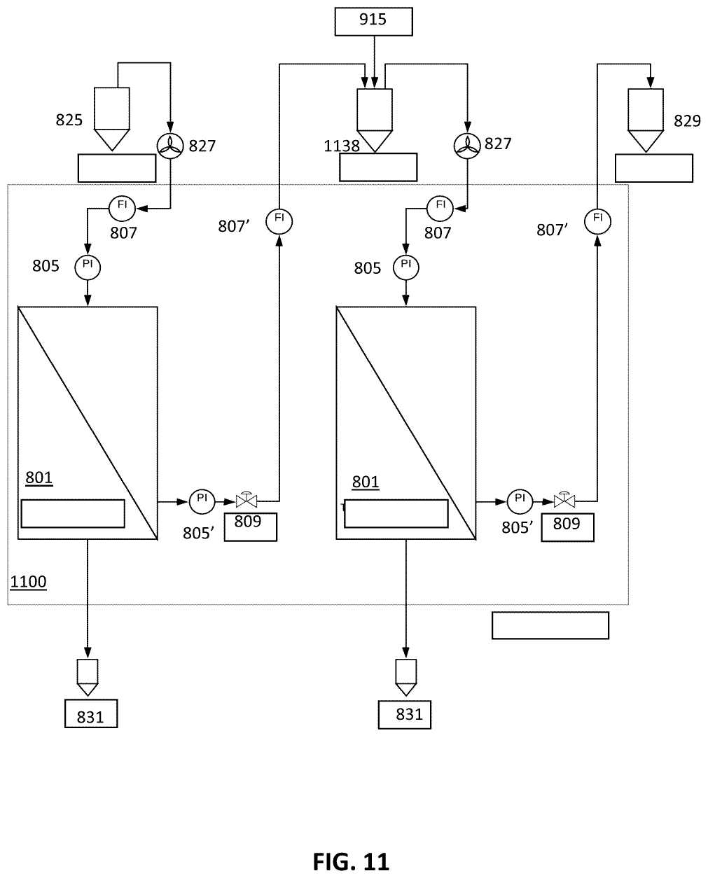

The variation shown in FIG. 11 is similar to that shown in FIG. 9 and allows the addition of new dilution buffer 915 to be added to the concentrated therapeutic product 1138 off of the chip before being fed back into the chip for further concentration.

Flow (of fluid) through each of the channels may be controlled. For example, flow through the feed channel may be maintained at a flow rate within a target range, e.g., of between about 0.5 mL/min and about 4 mL/min (e.g., about 2 mL/min), etc. The flow rate of the feed solution may drop along the length of the flow channel, since the solvent is being filtered out of the feed solution. The apparatus may be configured as described herein to maintain the flow rate within the target range. Further, any of the microfluidic concentrator and buffer exchange apparatuses described herein may maintain a constant sheer rate over the membrane (e.g., in the feed channel). The membrane may be prone to clogging due to the low shear rate. The sheer rate may be maintained within a target range. For example, in some examples, the sheer rate may be maintained within a target range. The sheer rate may be maintained at an approximately constant sheer rate over the membrane. In some examples, the dimensions of the channel (e.g., the feed channel) may be reduced along the length of the channel to maintain a constant shear rate over the membrane. The sheer rate γ in these apparatuses is proportional to the flow rate, Q (γ∝Q). The flow rate, Q, is related to the channel dimensions, including the channel height (h) and channel height (w). For example, Q (and therefore flow rate) is proportional to 1/h² and to 1/w.

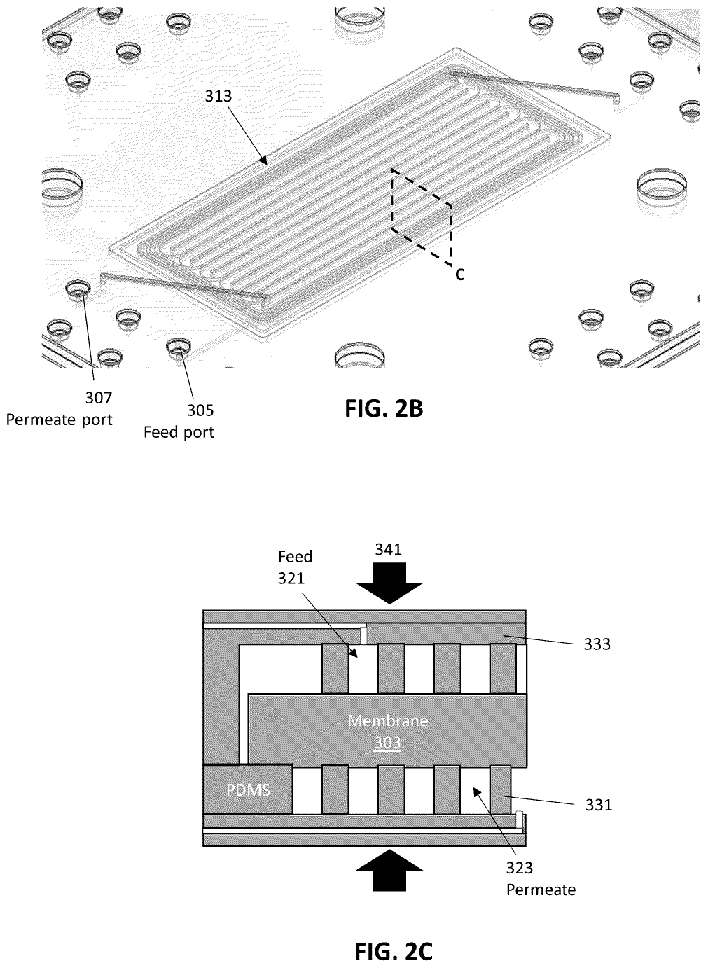

In some examples, the microfluidic concentrator and buffer exchange apparatus includes serpentine channels. For example, the feed channel, and/or permeate channel may be arranged in each layer in a back-and-forth layout that zigzags in the plane of the first, second and third layers. For example, FIG. 2A shows an example of a microfluidic concentrator apparatus 300 that is formed as a microfluidic chip including a microfluidic concentrator portion 313, as well as inlet and outlet ports for the feed (feed inlet port 305) and permeate (permeate inlet port 307). The apparatus may also include inlet and outlet ports for buffer. In some examples the apparatus also includes one or more pumps, one or more integrated sensors. FIG. 2B shows an enlarged view of the microfluidic concentrator portion 313 that is configured with the channels arranged in a serpentine pattern to allow them to be tightly packed in the layered microfluidic chip. A cross-section through a region C of the microfluidic concentrator portion in FIG. 2B is shown in FIG. 2C. As shown in FIG. 2C, this portion of the microfluidic concentrator includes the feed channel 321 that is adjacent to the permeate channel 323. The same channels are shown in cross-section multiple times, as they extend in a serpentine path. A first membrane 303 separates one side of the feed channel from an adjacent side of the permeate channel. The membrane may be sealed between the two layers (e.g., the first layer 331 forming the permeate channel and the second layer 333 forming the feed channel) by the application of a sealing force 341.

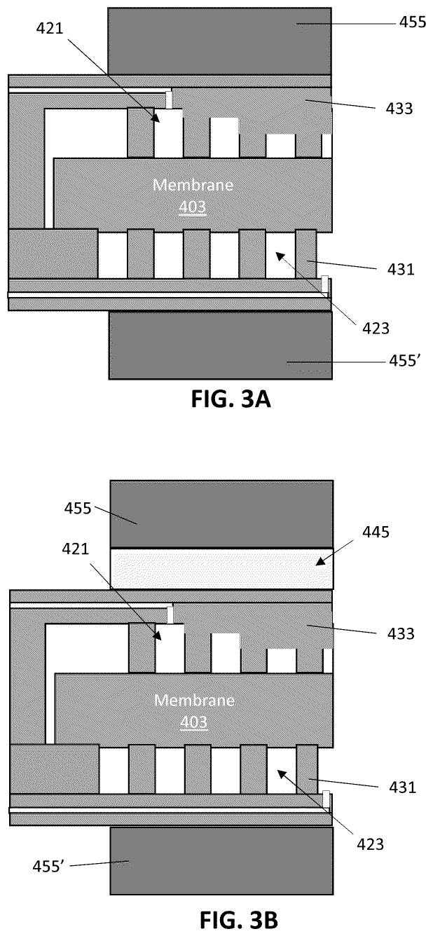

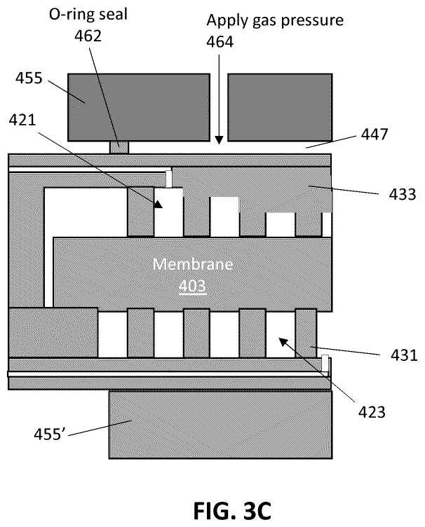

FIGS. 3A-3C illustrate examples of sealing structures that may be used to secure the membrane(s) of the microfluidic concentrator and buffer exchange apparatus between the feed channel and permeate channel and/or the buffer channel and feed channel. For example, FIG. 3A shows a frame 455, 455′ that may clamp the layers, e.g., the first layer 431 of the permeate channel 423 and second layer 433 of the feed channel 421 and the membrane 403 together to seal the membrane between the layers. The frame may be a polymeric and/or metal frame and may clamp the layers and membrane together.

In some examples, the membrane may be sealed along the length of the channel by sealing structure comprising a pressure-distributing seal that distributes the sealing force. This may reduce (and in some instances even prevent) leakage around the membrane(s) during operation, which may include operation under pressure. For example, FIG. 3B shows a schematic illustration of a first example in which a pressure-distributing seal 445 is included over the side of the feed channel that is opposite from the membrane. In this example the pressure-distributing seal is an expandable foam material that may distribute pressure uniformly across the top. The pressure-distributing seal may be on either side or both sides of the apparatus, e.g., between the frame 455, 455′ and the layers.

FIG. 3C illustrates another example of a sealing structure comprising a pressure-distributing seal 447 that is configured to use fluid pressure to distribute the clamping/sealing force to hold the membrane between the layers. In FIG. 3C, the frame 455, 455′ includes a channel into which fluid (e.g., gas, air, etc.) may be applied 464 under pressure. The pressure-distributing seal may include an O-ring 462 to secure the fluid pressure against the layers. Thus, during operation of the apparatus, a pressure port on the apparatus may be engaged with the frame so that pressure may be used to help seal the membrane(s) between the layers.

In some examples, the apparatuses described herein may be combined in parallel. For example, a system for concentrating and exchanging buffer may include a stack of multiple single pass, tangential flow concentrator and buffer exchange apparatuses as described herein.

In any of the single-pass concentrators described herein, the type of membrane may vary depending on the type of therapeutic and/or design constraints of the apparatus. In some examples, the membrane has a pore size that is a particular percentage less than the therapeutic particle size (e.g., average particle diameter). In some cases, the pore size is less than about 10% to about 25% of the of the therapeutic particle size (e.g., <10-25%, <15-25%, <10-20%, <20-25%, or <10-15% of the therapeutic particle size). Depending on the geometry, the term “size” here may refer to the diameter, length, width, etc., of a particle. In some examples, the membrane comprises a cellulose, a silicon-based, or an aluminum oxide-based material, or a combination of any of the foregoing. Example membranes may include Biomax™ Ultrafiltration Membrane, 500 kDa (PES) (MilliporeSigma™, U.S.), Biomax™ Ultrafiltration Membrane, 50-500 kDa (PES) (MilliporeSigma™, U.S.), Ultracel™ Ultrafiltration Membrane, 30-300 kDa (CRC) (MilliporeSigma™, U.S.), ProStream™ Membrane, 30-300 kDa (PES) (Repligen™, U.S.), and/or Hystream™ Membrane, 30-300 kDa (PES) (Repligen™, U.S.).

Apparatuses with Integrated Concentrator and Buffer Exchange Regions

Buffer exchange may be performed using an integrated concentration and buffer exchange portion through a second membrane (which may be positioned opposite from the first membrane). Buffer exchange may be done by removing old buffer from the therapeutic solution and adding new buffer to the retained therapeutic solution (the retentate) at the same time.

For example, the apparatus is, or may include an integrated concentrator and buffer exchange region. For example, the second concentrator of the apparatus may be an integrated concentrator and buffer exchange region, wherein the buffer input is fluidly connected with a buffer channel that extends in a second serpentine pathway in a third layer, wherein the buffer channel extends adjacent to a second feed channel that is in fluid communication with the first retentate output, and wherein the second feed channel is separated from a second permeate channel by a second membrane region on a first side of the feed channel, and the second feed channel is separated from the buffer channel by a third membrane region on a second side of the feed channel.

In apparatuses in which the second concentrator is an integrated concentrator and buffer exchange region, the cross-sectional area of buffer channel may decrease along the second serpentine pathway from the buffer input to a second retentate output.

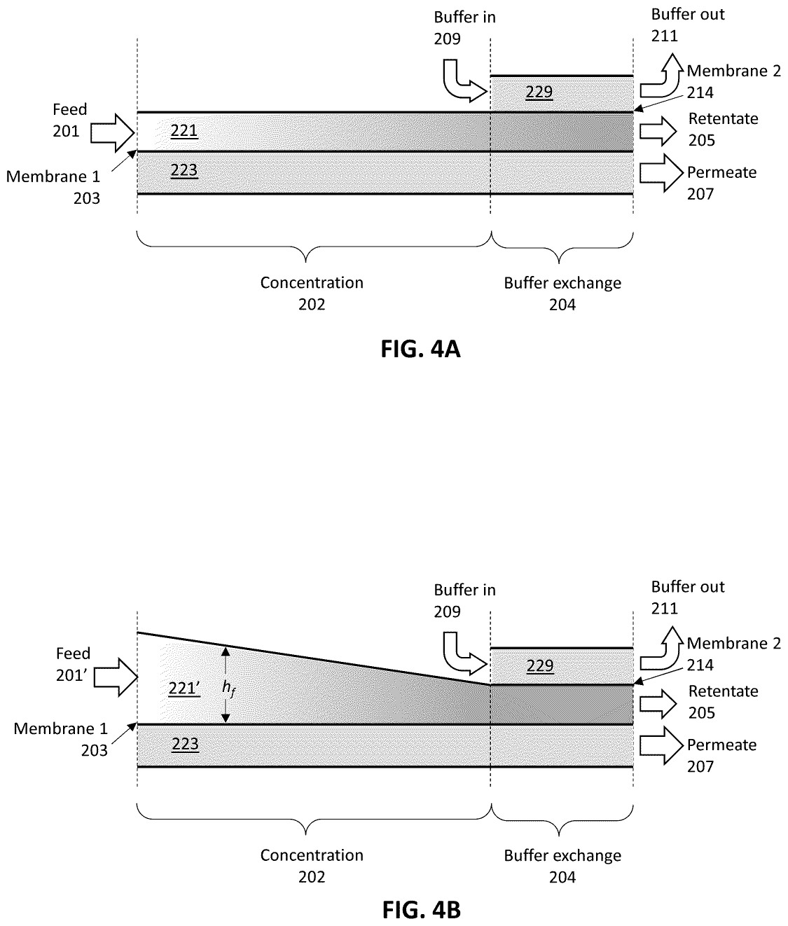

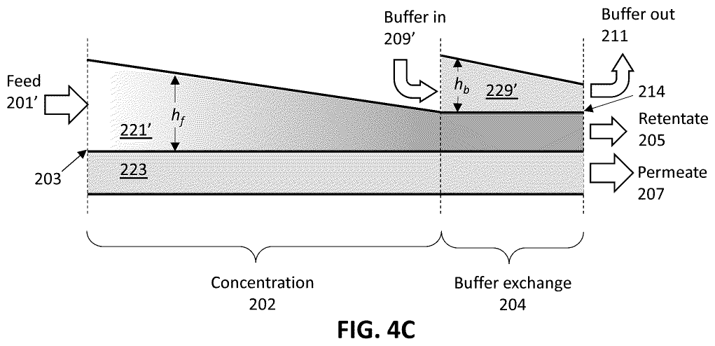

FIGS. 4A-4C illustrate another example in which the buffer dilution/buffer exchange and second concentrator are integrated together, and a second membrane separates the second feed channel region from a buffer channel so that the buffer is exchanged with the retained therapeutic solution through the second membrane. Processing through the microfluidic concentrator and buffer exchange apparatus proceeds as a single pass, in a single flow direction, without the need to alternate direction of buffer perfusion through the membrane.

In this example, as the therapeutic solution flows through the feed channel, the permeate solution may flow in the permeate channel in the same direction as the therapeutic solution in the feed channel; alternatively in some example the feed channel may flow in the opposite direction. Similarly, the buffer solution in the buffer channel may flow in the same direction as the feed channel; alternatively in some examples the buffer solution may flow in the opposite direction.

In examples in which the upstream buffer exchange and concentration are combined, the apparatus may include a first membrane (e.g., ultrafiltration membrane) that separates a feed channel, which may also be referred to as a feed-retentate channel, from a permeate channel.

In addition, a second region of the microfluidic concentrator and buffer exchange apparatus exposes the feed solution to a new buffer solution through a second membrane. As the retentate feed solution passes through this portion of the microfluidic concentrator and buffer exchange apparatus, new buffer material is added to the therapeutic solution while the original buffer material may be removed, e.g., into the permeate channel.

The feed channel, and in particular the portion of the feed channel that is adjacent to the permeate channel, may be about 5 meters or longer (e.g., about 6 meters or longer, about 7 meters or longer, about 8 meters or longer, or more.). These channels may be relatively long and relatively narrow, and arranged in a relatively compact, serpentine path. The permeate channel may mirror the feed channel. The buffer channel may extend a portion (e.g., sub portion or subregion) of the feed channel. As mentioned, the microfluidic concentrator and buffer exchange apparatuses described herein may regulate the flow rate within the feed channel, permeate channel and/or the buffer channel. Flow sensors may be configured to measure flow into the permeate channel, out of the permeate channel, into the buffer channel and/or out of the buffer channel.

FIGS. 4A-4C schematically illustrate microfluidic concentrator and buffer exchange apparatuses. In FIG. 4A, the apparatus schematic illustrates a concentration region 202 and a buffer exchange region 204. In this example the feed channel 221 receive the therapeutic solution into the feed port 201 and exits at a feed outlet 205. A permeate channel 223 flows adjacent to the feed channel and is separated by a first membrane 203 through which small molecules (e.g., ethanol, salts, etc.) pass in the permeate solution to exit from the channel at the permeate outlet 207. The feed channel and the permeate channel may extend a length, L, in a serpentine path (not shown) that may be tightly packed in the microfluidic apparatus (e.g., microfluidic chip). The feed channel may be part of a second layer that extends adjacent to a first layer; the permeate channel may be in the first layer. The first membrane may be sandwiched and sealed between the first and second layers. The concentrator region in FIG. 4A is shown extending a portion of the full length of the apparatus, a second, subsection, of the length of the feed channel includes a second membrane 214 separating a buffer channel 229. The buffer channel receives a buffer solution in the buffer inlet 209 that flows adjacent to this subsection of the feed channel, separated by the second membrane. The buffer channel also includes a buffer outlet 211. Sensors may monitor flow and/or pressure (not shown), as mentioned above.

In FIG. 4A, the feed channel may have a constant diameter along its length. In FIG. 4B the height, h_{f}, of the feed channel 221′ decreases along the initial portion of the length, as schematically illustrated, which may increase the sheer rate along the length. The rate of change of the height may be constant (as shown) or non-constant. For example, the rate of change may be greater initially, but may decrease over the length of the feed channel. The rate of change of the height may be matched to the desired sheer rate so that the sheer rate may be held approximately constant during operation of the apparatus. The height of the feed channel may be adjusted on the side of the feed channel that is opposite the first membrane (e.g., a region that is not adjacent to the buffer channel). In some examples the width of the feed channel may also be decreased along the length. Alternatively, in some examples, just the height or just the width is decreased to keep a relatively constant sheer rate. In variations in which the width is changed, the region of the feed channel that contacts the permeate channel may remain constant. For example, the channel may be rounded on at least the side closest to the permeate channel, so that the width (the distance between the sides of the channel) may be changed without changing the width of the contact region. Alternatively, in some examples the width of the contact region (the membrane) between the channels may be changed without changing the width of the channels, e.g., by exposing or occluding more or less of the membrane between the channels.

FIG. 4C illustrates and example in which the height, h_{b}, of the buffer channel 229′ decreases over its length. The height of the feed channel, h_{f}, in this example also decreases. In some examples the height of the buffer channel may vary (e.g., decrease) while the height of the feed channel may remain relatively constant.

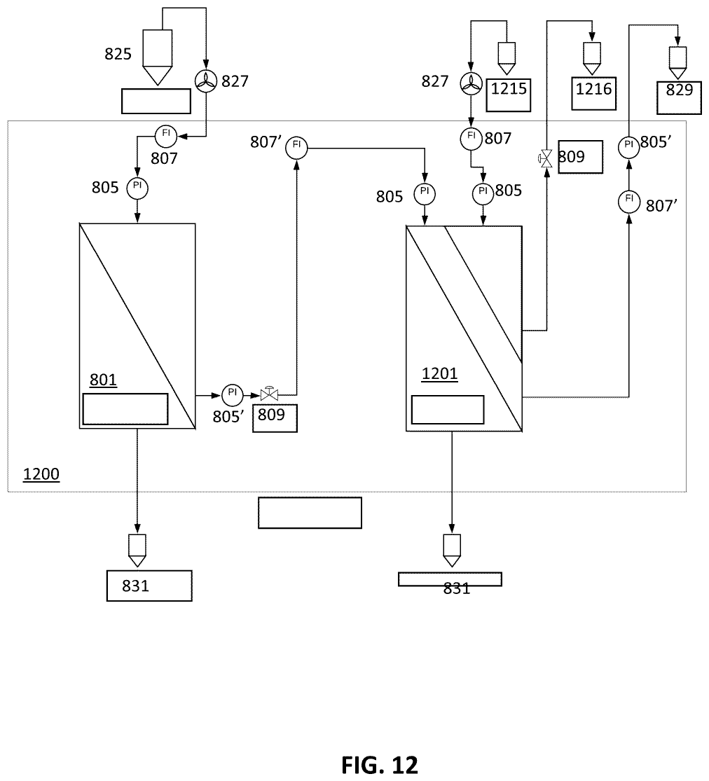

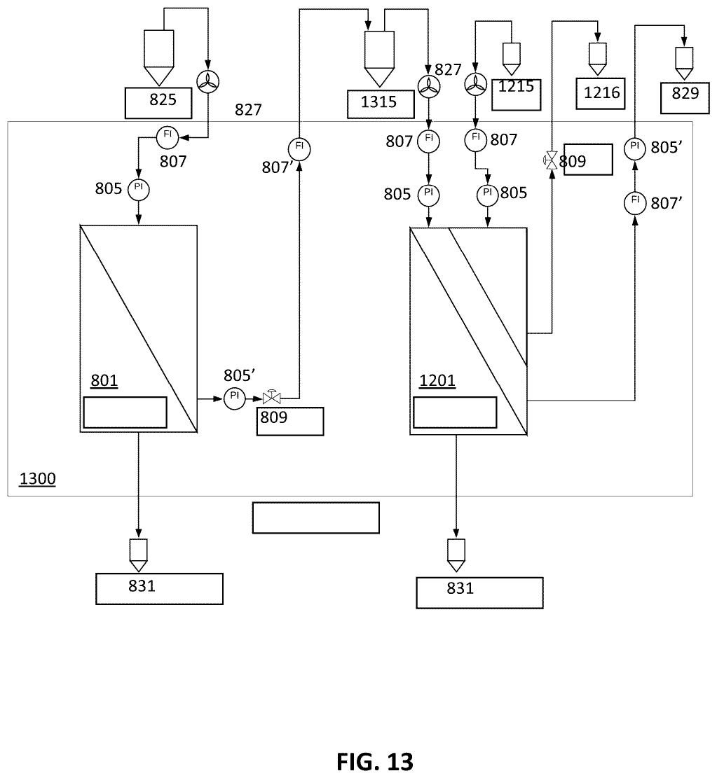

FIGS. 12 and 13 schematically illustrate examples of apparatuses including integrated buffer exchange modules. In FIG. 12, the apparatus 1200, which may be configured as a chip and/or as a cartridge including such a chip, includes product (e.g., therapeutic) input to which a pressure sensor 805 and a flow sensor 807 are coupled. The raw therapeutic product 825 is fed into the concentrator module 801; in some examples this concentrator module is integrated with a buffer exchange module 1201 in the chip. A buffer input material 1215 may pass through the buffer exchange module to a metered (by control valve 80) buffer output 1216. The output of the buffer exchange module retentate is the final therapeutic product output 829. The integrated concentrator and buffer exchange region may be referred to herein as an integrated module. FIG. 13 shows a similar example to that shown in FIG. 12, but the output of the concentrator (concentrated drug output 1315) may be stored off of the chip 1300 before being fed into the buffer exchange module 1201.

In any of the apparatuses described herein, the degree of buffer exchange in the apparatus may be increased by, for example, increasing the length of the buffer exchange section of the apparatus. In some examples, the degree of buffer exchange may be increased by adding multiple buffer exchange modules in series as part of the system. For example, if each buffer exchange module is able to reduce the concentrator of a solvent, such as ethanol, from the buffer by, e.g., 10×, having three such modules connected in series will reduce the solvent (e.g., ethanol) concentration by 1000×. Thus, any of these apparatuses may achieve different solvent reduction level by adding different number of modules based on different applications/therapies.

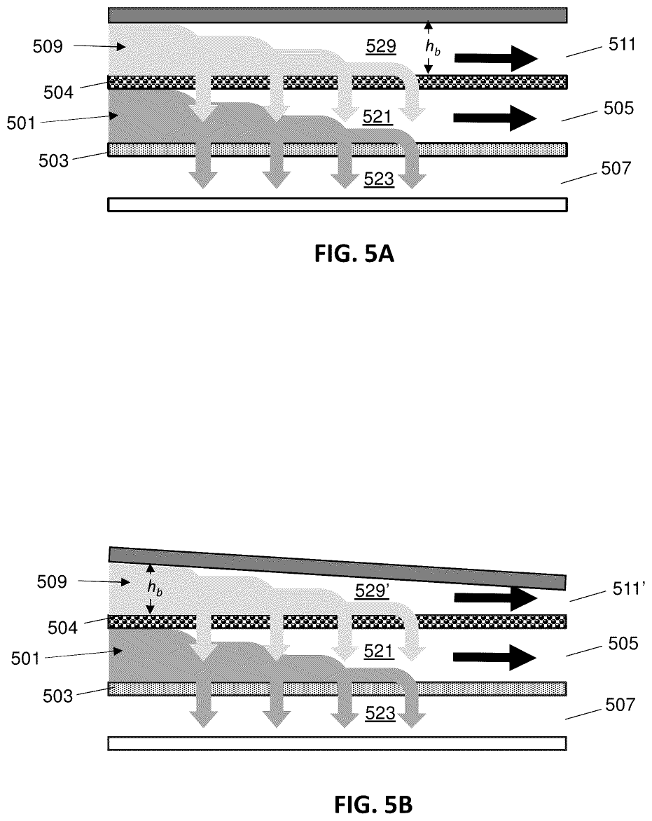

The microfluidic concentrator and buffer exchange apparatuses described herein may include a buffer exchange region, which may also be referred to as a buffer exchange module, over a portion of the length of the feed channel. FIGS. 5A and 5B illustrate the operation of this region. In FIG. 5A, the buffer channel has a constant height, h_{b}, while in FIG. 5B, the height of the feed channel decreases along the length of the feed channel, as described above. An exchange buffer solution 509 is added into the buffer channel 529 and exits the buffer channel 529 at a buffer outlet port 511. The therapeutic solution enters the feed channel 521 through a feed input 501, and the retentate exits the feed channel from a feed outlet (retentate outlet) port 505. Permeate solution travels within the permeate channel 523 and exits at a permeate outlet 507.

In one example, the therapeutic particle concentration is set to be approximately the same in the feed input as it is at the feed (retentate) output, and the flow rate along the channel may be uniform (Q_{feed}=Qᵣₑₜₑₙₜₐₜₑ). In general, the flow rate within the feed channel, buffer channel and permeate channel may be controlled by one or more pumps (e.g., pressure pump, peristaltic pump, syringe pump, etc.). In FIG. 5A, new buffer is added to the therapeutic solution in the feed channel through the second membrane (filter membrane 504). Old buffer will be forced into the permeate channel through the first membrane (filter membrane 503). The degree of buffer exchange may be determined by the new buffer addition volume, channel length, and/or flow rate.

In FIG. 5B, the volume of the buffer channel 529′ may be adjusted by decreasing the height, h_{b}, of the buffer channel along the length of the buffer channel to the buffer output 511′. In general, the flow rate of the buffer change may be adjusted by adjusting the channel resistance; decreasing the channel height may increase the channel resistance. In this example, channel resistance may be adjusted so that buffer is added into the therapeutic solution at a constant rate. The buffer exchange portion of the microfluidic concentrator and buffer exchange apparatus is part of the single-pass fluid pathway. In FIG. 5B, the buffer channel dimensions control the exchange buffer to feed rate (which is the same as the feed rate to permeate rate in this example). For example, if there is a constant flow of permeate material from out of the feed channel into the retentate channel (e.g., the apparatus is operated in a saturation region in which transmembrane flux is no changed with the transmembrane pressure), the flow of therapeutic solution through the feed channel may be maintained at an approximately constant rate if the flow rate from the buffer channel into the feed channel is equal to the flow rate from the feed channel into the permeate channel. This may be achieved with a constant pressure drop between the exchange buffer and the feed channel. For example, the feed channel may have a constant depth and width while the cross-sectional volume of the buffer channel is decreased (e.g., by decreasing the depth with a constant width, or by decreasing the width with a constant depth, or by decreasing both depth and width, while maintaining the membrane surface area along the length between the channels the same) to maintain a constant pressure drop between the exchange buffer and the feed channel.

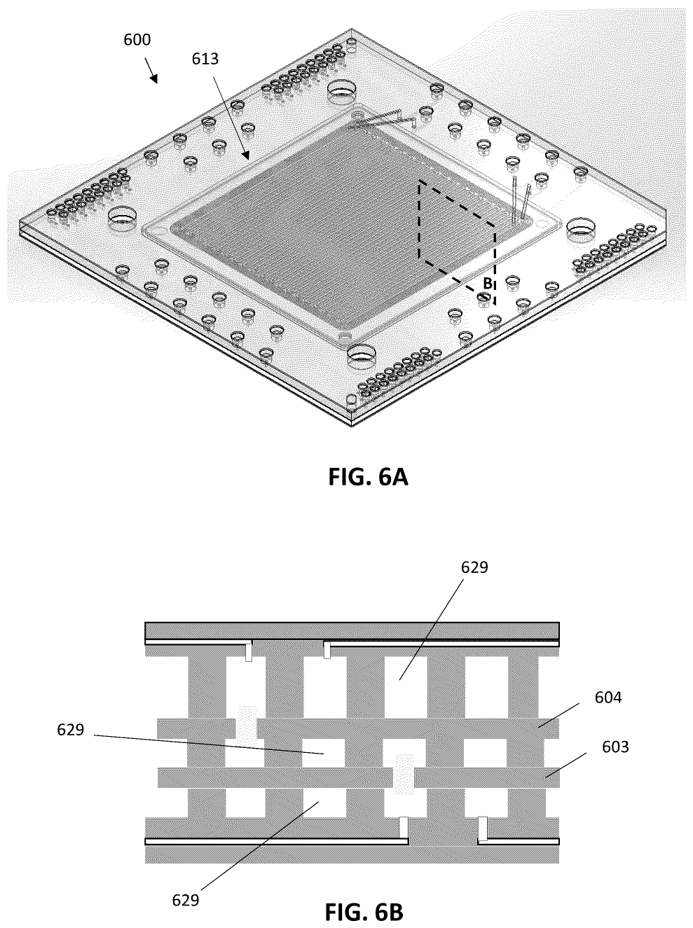

FIG. 6A illustrates another example of a microfluidic concentrator and buffer exchange apparatus configured as a microfluidic chip. The apparatus includes a microfluidic concentrator and buffer exchange region 613, as well as inlet and outlet ports. FIG. 6B shows an enlarged view of a section through a region (“B”) of the apparatus of FIG. 6B, showing the feed channel 621, permeate channel 623 and buffer channel 629, separated by the first membrane 603 and the second membrane 604, as described above.

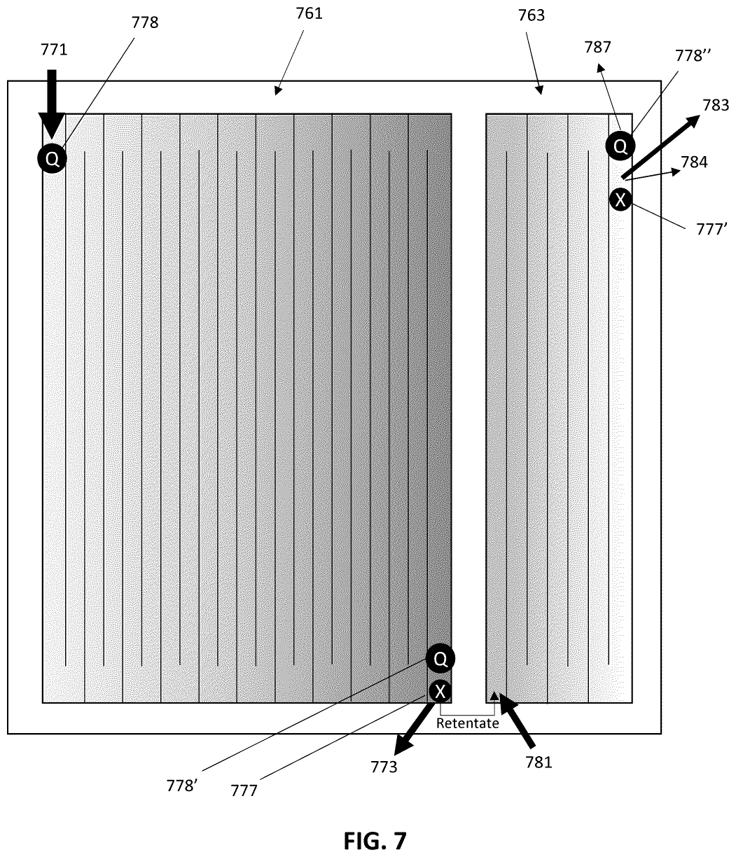

FIG. 7 illustrates one example of a single pass, tangential flow concentrator and buffer exchange apparatus, shown as a microfluidic chip. In this example the channels are serpentine. The concentrator-only region 761 of the microfluidic concentrator and buffer exchange apparatus is on the left side of the apparatus, while the concentrator and buffer exchange region 763 is on the right side of the apparatus. The apparatus also includes a feed inlet 771 that may include a flow sensor 778, such as an on-chip flow meter. The therapeutic solution may be added into the microfluidic concentrator and buffer exchange apparatus from the feed port the apparatus includes a valve 777 for releasing permeate from a permeate port 773 midway along the apparatus, before the buffer exchange region. A flow meter 778′ may also be included at this region. The portion of the therapeutic solution (retentate) passing into the buffer exchange region may be passed into the buffer exchange portion (note that concentration may also be occurring at the buffer exchange portion, as the permeate channel also extends adjacent to the feed channel in this region). In FIG. 7, the inlet port for the buffer 781 is shown at the start of the buffer exchange region. The buffer solution may leave the buffer channel at the buffer outlet 783. The example shown in FIG. 7 also illustrates the retentate outlet 787 and the permeate outlet 784. One or more valves 777′ and/or sensors (e.g., flow sensors) 778″ may also be included at or near the outlet ports, e.g., for sensing and/or controlling flow in the feed channel. The flow meter(s) and valves may be used to keep the flow rate at or within a target range. All of the methods and apparatuses described herein, in any combination, are herein contemplated and can be used to achieve the benefits as described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the apparatus in use or operation in addition to the orientation depicted in the figures. For example, if an apparatus in the figures is inverted, elements described as “under” or “beneath” other elements or features would then be oriented “over” the other elements or features. Thus, the exemplary term “under” can encompass both an orientation of over and under. The apparatus may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

1. A microfluidic apparatus, the apparatus comprising:
a first concentrator region comprising:
a first permeate channel that extends in a first serpentine pathway in a first layer,
a first feed channel that extends in the first serpentine pathway in a second layer from a feed input to a first retentate output, wherein the first permeate channel extends adjacent to the first feed channel, and
a first membrane region that separates the first permeate channel from the first feed channel;
a buffer input downstream the first retentate output for adding buffer into a retentate leaving the first feed channel; and
a second concentrator region in fluid communication with the first retentate output.

2. The apparatus of claim 1, wherein a cross-sectional area of first feed channel decreases along the first serpentine pathway from the feed input to the first retentate output to maintain a constant shear rate within the first feed channel.

3. The apparatus of claim 1, wherein a height of the first feed channel shortens along the first serpentine pathway from the feed input to the first retentate output.

4. The apparatus of claim 1, further comprising a pressure-distributing seal that distributes pressure to seal the first membrane region between the first layer and the second layer.

5. The apparatus of claim 4, wherein the pressure-distributing seal comprises a sheet of compressive foam.

6. The apparatus of claim 4, wherein the pressure-distributing seal comprises a pressurizing chamber.

7. The apparatus of claim 1, wherein the first serpentine pathway has a length of greater than about 5 meters.

8. The apparatus of claim 1, wherein the first membrane region has an area of greater than about 50 cm2.

9. The apparatus of claim 1, wherein the first membrane region comprises a Polyethersulfone (PES), a Composite Regenerated Cellulose (CRC) membrane, or a combination thereof.

10. The apparatus of claim 1, wherein the first membrane region is permeable to ethanol.

11. The apparatus of claim 1, further comprising a feed input port fluidly connected with the feed input and a retentate output port fluidly connected with the second concentrator region.

12. The apparatus of claim 1, wherein the buffer input is fluidly connected with a buffer channel that extends in a second serpentine pathway in a third layer, wherein the buffer channel extends adjacent to a second feed channel that is in fluid communication with the first retentate output, and wherein the second feed channel is separated from a second permeate channel by a second membrane region on a first side of the second feed channel, and the second feed channel is separated from the buffer channel by a third membrane region on a second side of the second feed channel.

13. The apparatus of claim 12, wherein the cross-sectional area of buffer channel decreases along the second serpentine pathway from the buffer input to a second retentate output.

14. An apparatus comprising:
a first concentrator region comprising:
a first permeate channel that extends in a first serpentine pathway in a first layer,
a first feed channel that extends in the first serpentine pathway in a second layer from a feed input to a first retentate output, wherein the first permeate channel extends adjacent to the first feed channel, and
a first membrane region that separates the first permeate channel from the first feed channel;
a dilution buffer region in fluid communication with the first retentate output of the first feed channel; and
a dilution buffer input into the dilution buffer region; and
a second concentrator region comprising:
a second permeate channel that extends in a second serpentine pathway in the first layer,
a second feed channel in fluid communication with an output of the dilution buffer region, wherein the second feed channel extends in the second serpentine pathway in the second layer, wherein the second permeate channel extends adjacent to the second feed channel, and
a second membrane region that separates the first permeate channel from the second feed channel; and
a second retentate output in fluid communication with the second feed channel.

15. The apparatus of claim 14, wherein a cross-sectional area of first feed channel decreases along the first serpentine pathway from the feed input to the first retentate output to maintain a constant shear rate within the first feed channel.

16. (canceled)

17. The apparatus of claim 1, wherein a height of the first feed channel shortens along the first serpentine pathway from the feed input to the first retentate output and wherein a height of the second feed channel shortens along the second serpentine pathway from an output of the dilution buffer region to the second retentate output.

18. The apparatus of claim 1, wherein the first membrane region and the second membrane region are parts of a single membrane.

19.-25. (canceled)

26. The apparatus of claim 1, further comprising a feed input port fluidly connected with the feed input and a retentate output port fluidly connected with the second retentate output.

27. The apparatus of claim 1, further comprising a permeate output out of the first permeate channel at approximately the dilution buffer input.

28. A method comprising:
passing a therapeutic polynucleotide solution through a first feed channel having a first serpentine length in a first region of a microfluidic apparatus;
filtering small molecules out of the therapeutic polynucleotide solution out of the first feed channel by tangential flow filtration into a permeate channel adjacent to the first feed channel through a first membrane region while maintaining a constant shear rate relative to the first membrane region as the therapeutic polynucleotide solution passes through the first serpentine length of the first feed channel; and
adding a buffer solution into the therapeutic polynucleotide solution after the first feed channel and concentrating the therapeutic polynucleotide solution in a second region of the apparatus.

29.-61. (canceled)
